(19) United States
(12) Patent Application Publication
(10) Pub. No.: US 2009/0210066 A1
(43) Pub. Date: Aug. 20, 2009

(54) ANTERIOR CRUCIATE LIGAMENT SUBSTITUTING KNEE REPLACEMENT PROSTHESIS

(75) Inventor: Murali Jasty, Weston, MA (US)

Correspondence Address: PROSKAUER ROSE LLP, 1001 PENNSYLVANIA AVE, N.W.,, SUITE 400 SOUTH, WASHINGTON, DC, 20004, US

(73) Assignee: The General Hospital Corporation dba Massachusetts General Hospital, Boston, MA (US)

(21) Appl. No.: 10/597,364

(22) PCT Filed: Jan. 21, 2005
(86) PCT No.: PCT/US05/01888

§ 371 Date: Aug. 19, 2008

Related U.S. Application Data
(60) Provisional application No. 60/538,228, filed on Jan. 23, 2004.

(51) Int. Cl. A61F 2/38

(52) U.S. Cl. 623/20.31; 623/20.21

(57) ABSTRACT

There is disclosed a total knee replacement prosthesis, which can substitute the function of an anterior and/or a posterior cruciate ligament. A femoral component containing two intercondylar surfaces and an intercondylar region, a tibial component having a tibial platform and a bearing component, and a protrusion from the bearing component also are disclosed.

This application claims priority to U.S. provisional application Ser. No. 60/538,228, filed Jan. 23, 2004, the entirety of which is hereby incorporated by reference.

### BACKGROUND OF THE INVENTION

1. Field of the Invention

The present invention relates to knee replacement prosthesis. More specifically, the invention pertains to prosthetic knee implants, which are implanted in the absence of a functional anterior cruciate ligament and provide a substitute for the function of the anterior and/or a posterior cruciate ligament.

2. Background of the Invention

The natural knee joint is complemented by two collateral ligaments, one on the lateral side of the joint and the other on the medial side thereof, each attached both to the tibia and to the femur. The points of attachment of the collateral ligaments to the femur are approximately on the axis of the arc along which the other end of the tibia moves and the knee flexes. In addition to the two collateral ligaments on the outsides of the knee joint, there also are two cruciate ligaments in the middle of the knee joint. One of these cruciate ligaments is attached to the posterior margin of the tibia, while the other is attached towards the anterior margin of the tibia. Both ligaments are attached to the femur in the notch between the condyles approximately on the axis of the collateral ligaments. Often one or both of the cruciate ligaments, particularly the anterior cruciate ligament, deteriorate as a result of the degeneration of the knee joint, which gives rise to the need for a knee prosthesis implantation operation. Hence, the surgeon may remove the anterior cruciate ligament, or both of the cruciate ligaments, in the course of the implantation operation.

The absence of the normal function of an anterior cruciate ligament leads to alteration in the gait and other functional activities of the total knee replacement patients and decreases the strength of the muscles about the knee. Many recent studies have shown that total knees without functioning anterior cruciate ligament move in an abnormal fashion with the tibial femoral contact areas lying more posteriorly in full extension than in the normal intact knee with functioning anterior cruciate ligament and moving anteriorly in paradoxical fashion with further knee flexion (see for example, Komistek et al., J Arthroplasty 17(2):209-216, 2002). These positions and movements which are the reverse to those occurring in normal knee produce abnormal knee kinematics, which can lead to alterations in the gait and functional activities of the patients who often report difficulties with activities such as stair descent. Furthermore, these alterations also decrease the efficiency of the quadriceps mechanism which decreases the strength of the knee.

The prosthetic knee also is subjected to excessive wear due to large amounts of sliding between the femoral and tibial bearing surfaces, which compromises the longevity of the total knee replacements. The tibial component is also subjected to abnormal rocking stresses due to the deviation of the tibial femoral contact points anteriorly and posteriorly from the midline during gait.

Knee prostheses, as known in the art (for example, U.S. Pat. No. 6,264,697), have guided surfaces throughout the range of motion for control of anterior-posterior displacement of the tibia. While this appear beneficial, in reality the motion is determined by the remaining of collateral and cruciate ligaments. Any attempts to control this sliding motion, through out the range of flexion by guided surfaces, would be difficult.

Previously known knee prosthesis contains tibial guide surface, which has an anterior and posterior upward sweep, which engages in recesses in the femoral component to contribute stability. Thus, the middle surface of the guide surface is concave, when viewed from the top, with projections on the anteriorly and posteriorly surfaces with articulating surfaces on the posterior and anterior aspect, respectively. This would create abnormally high forces, which would tend to cause tilting of the liner; therefore, the tray at terminal extension and flexion, when the femoral cam contacts the anterior most and posterior most aspects of the tibial liner. Because, the contact areas are far from the midline of the tibial component. These tilting forces can cause premature loosening of the tibial component or breakage or disengage of the liner from the tray.

A cam on the femoral side engages the guide surface on the tibia or a guide surface which connects the medial and lateral condyles of the femur. The space required to put the cam or the guide surface which extends all the way posteriorly results in excessive removal of the femoral bone in the intercondylar region.

Knee replacement prosthesis can provide a substitute for the function of an anterior cruciate ligament, particularly in cases where a knee joint has ceased to function as a result of deformative joint disorders, rheumatism, or external injury, etc. Prior to the present invention, currently available knee replacement prostheses are substantially comprised of a femoral component in which two protruding surfaces, i.e., medial and lateral protruding surfaces, are joined in a front and back relationship to form a femoral condylar portion, and a tibial component. Recessed surfaces in the tibial component support the femoral condylar portion so that the femoral condylar portion is capable of a sliding movement. A rolling movement are joined in a front and back relationship to form a tibial condylar portion. The femoral condylar portion, in this case, has a medial condylar section and a lateral condylar section, and both of these portions are formed so that the trajectory connecting the lowest points of the two portions constitutes an approximate circular-arc curve in two dimensions. In a conventional prosthetic knee, imaginary extended lines of this approximate circular-arc curve in the anteroposterior direction are set parallel to each other. This parallel setting sets limitations on the region of possible movement of the prosthetic knee. Therefore, it is difficult to achieve maximum flexion with such approaches.

Also, currently available total knee replacement prostheses implants generally require the sacrifice of ligaments and natural bone in order to accommodate the mechanism which attempts to drive and contain the replacement knee in a more normal fashion. The mechanism usually includes a prominent eminence on the tibial component and a relatively large recess in the femur to accommodate the eminence. Such replacement prostheses thus require more radical surgery and increase the shear stresses encountered at the interface between the implant and the natural bone.

Total knee replacements provide dramatic relief of pain and improvement of functions for patients with end stage arthritis of joints. However, most of the currently available prosthetic knee implants employed for the total replacement of the natural knee joint do not adequately replicate the function of the anterior cruciate ligament, which is either absent prior to the replacement procedure or is sacrificed during the procedure. In contrast, the posterior cruciate ligament is often present regardless of the extent of the arthrosis and great care is exercised either preserve the function of the posterior cruciate ligament during the replacement procedure or substitute its function by specific features in the design of the prosthetic components.

Several US patents describe various aspects of artificial knee joint prosthesis and significance of cruciate ligaments function (see for example, U.S. Pat. Nos. 5,413,604; 5,358,527; 6,406,497; and 6,342,075). Several other US patents describe various components of knee joint including femoral and tibial (see for example, U.S. Pat. Nos. 5,658,342; 5,935,173; 6,074,425; 6,558,421; 5,219,362; 4,216,549; 6,080,195; 6,413,279; and 5,011,496) components. Various US patents also disclosed total knee replacement prosthesis which flexes to a complete flexion of up to 130° (see for example, U.S. Pat. Nos. 6,264,697; 4,959,071; 5,147,405; 6,190,415; 5,282,869; 5,997,577; and 6,152,960).

However, until the instant invention, none disclosed a total knee replacement prosthesis, which can provide a substitute for the function of the cruciate ligaments, including the function of an anterior and/or a posterior cruciate ligament.

### SUMMARY OF THE INVENTION

One aspect of the invention provides to knee replacement prostheses, wherein the prostheses comprise a femoral component having a pair of condylar surfaces and an intercondylar region; and a tibial component having a tibial platform and a bearing component, such as a non-mobile or a mobile bearing, which articulate with the femoral component, wherein a protrusion or a tibial post from the bearing component articulates with the intercondylar portion of the femoral component. The prostheses, if desired, can provide substitute for the function of the cruciate ligaments, including the function of an anterior and/or a posterior cruciate ligament.

Another aspect of the invention provides knee replacement prostheses, wherein the prostheses comprise a femoral component having a pair of condylar surfaces and an intercondylar region; and a tibial component having a tibial platform and a bearing component, such as a non-mobile or a mobile bearing, which articulate with the femoral component, wherein a protrusion or a tibial post from the bearing component articulates with the intercondylar portion of the femoral component, wherein the tibial post is substantially curved in the sagittal plane to allow anterior-posterior translation of the femoral component during extension and early flexion, wherein anterior and posterior surfaces of the post is curved to allow and control femoral-tibial axial rotation. The prostheses, if desired, can provide substitute for the function of the cruciate ligaments, including the function of an anterior and/or a posterior cruciate ligament.

According to one aspect of the invention, the post can be anywhere from front to back of the tibial component. The anterior surface of the post is substantially curved in the sagittaly plane to allow anterior translation of the femoral component during extension and early flexion. The anterior surface of the post is offset from the main coronal plane of the post by 0 to 20 degrees to control femoral component rotation in extension. The anterior surface of the femoral component contacts the anterior surface of the post in extension and early flexion. The flexion is between about 0 to about 20 degrees.

According to another aspect of the invention, the posterior surface of the post is substantially curved in the sagittaly plane to allow posterior translation of the femoral component during late flexion. The posterior surface of the femoral component contacts the posterior surface of the post in late flexion. The flexion is between about 80 to about 150 degrees. The posterior surface of the post is substantially curved in the coronal plane to allow femoral component internal and external rotation. The posterior surface of the post is offset from the main coronal plane of the post by about 0 to about 20 degrees to control femoral component rotation in flexion.

In another aspect, the invention provides a knee replacement prosthesis, wherein the prosthesis comprises a femoral component having a pair of condylar surfaces and an intercondylar region; and a tibial component having a tibial platform and a bearing component, such as a non-mobile or a mobile bearing, which articulate with the femoral component, wherein a protrusion or a tibial post from the bearing component articulates with the intercondylar portion of the femoral component, wherein the tibial post is substantially curved in the sagittal plane to allow anterior-posterior translation of the femoral component during extension and early flexion, wherein anterior surface of the post is curved medial laterally to allow femoral-tibial axial rotation, wherein the femoral and tibial components are shaped in such a way that the femoral intercondylar surface has a radius of curvature at its distal most aspect which is slightly smaller than the radius of curvature of the anterior surface of the tibial projection, thereby providing a camming action, wherein the anterior articular surface of the tibial component is curved with a radius of curvature of the condylar surfaces which are about the same radius of curvature or slightly larger radius of curvature of the corresponding anterior condyles of the femoral component. The prosthesis, if desired, can provide a substitute for the function of the cruciate ligaments, including the function of an anterior and/or a posterior cruciate ligament.

According to one aspect of the invention, the anterior surface of the post is offset from the main coronal plane of the post by 0 to 20 degrees to control femoral component rotation in extension. The post can be anywhere from front to back of the tibial component. The anterior surface of the post is substantially curved in the sagittaly plane to allow anterior translation of the femoral component during extension and early flexion.

According to another aspect of the invention, the posterior surface of the post is substantially curved in the sagittaly plane to allow posterior translation of the femoral component during late flexion. The posterior surface of the femoral component contacts the posterior surface of the post in late flexion, wherein the flexion is between about 80 to about 150 degrees. The posterior-surface of the post is substantially curved in the coronal plane to allow femoral component internal and external rotation. The posterior surface of the post is offset from the main coronal plane of the post by about 0 to about 20 degrees to control femoral component rotation in flexion.

Yet in another aspect, the invention provides a method of repairing a damaged knee of a patient in need by implanting a total knee replacement prosthesis comprising the steps of:
(a) providing a femoral component having a pair of condylar surfaces and an intercondylar region; and
(b) providing a tibial component having a tibial platform and a bearing component, such as a non-mobile or a mobile bearing, which articulate with the femoral component, wherein a protrusion or a tibial post from the bearing component articulates with the intercondylar portion of the femoral component, wherein the tibial post is substantially curved in the sagittal plane to allow anterior translation of the femoral component during extension and early flexion, wherein anterior surface of the post is curved medial laterally to allow femoral-tibial axial rotation, wherein the femoral and tibial components are shaped in such a way that the femoral intercondylar surface has a radius of curvature at its distal most aspect which is slightly smaller than the radius of curvature of the anterior surface of the tibial projection, thereby providing a camming action, wherein the anterior articular surface of the tibial component is curved with a radius of curvature of the condylar surfaces which are about the same radius of curvature or slightly larger radius of curvature of the corresponding anterior condyles of the femoral component, thereby providing a total knee replacement prosthesis. The prosthesis, if desired, can provide a substitute for the function of the cruciate ligaments, including the function of an anterior and/or a posterior cruciate ligament.

According to one aspect, the invention provides methods of repairing a damaged knee of a patient in need by implanting a total knee replacement prosthesis, wherein the anterior surface of the post is offset from the main coronal plane of the post by 0 to 20 degrees to control femoral component rotation in extension, wherein the post can be anywhere from front to back of the tibial component, wherein the anterior surface of the post is substantially curved in the sagittaly plane to allow anterior translation of the femoral component during extension and early flexion, wherein the anterior surface of the femoral component contacts the anterior surface of the post in extension and early flexion, and wherein the flexion is between about 0 to about 20 degrees.

According to another aspect, the invention provides methods of repairing a damaged knee of a patient in need by implanting a total knee replacement prosthesis, wherein posterior surface of the post is substantially curved in the sagittaly plane to allow posterior translation of the femoral component during late flexion, wherein the posterior surface of the femoral component contacts the posterior surface of the post in late flexion, wherein the flexion is between about 80 to about 150 degrees, wherein the posterior surface of the post is substantially curved in the coronal plane to allow femoral component internal and external rotation, wherein the posterior surface of the post is offset from the main coronal plane of the post by about 0 to about 20 degrees to control femoral component rotation in flexion.

Still yet in another aspect, the invention provides a method of making a total knee replacement prosthesis comprising:
(a) obtaining a femoral component having a pair of condylar surfaces and an intercondylar region;
(b) obtaining a tibial component having a tibial platform and a bearing component;
(c) articulating the tibial platform and the bearing component with the femoral component;
(d) articulating a protrusion or a tibial post from the bearing component with the intercondylar portion of the femoral component;
(e) shaping the femoral and tibial components in such a way that the femoral intercondylar surface has a radius of curvature at its distal most aspect which is slightly smaller than the radius of curvature of the anterior surface of the tibial projection, thereby providing a camming action; and
(f) curving the anterior articular surface of the tibial component with a radius of curvature of the condylar surfaces which are about the same radius of curvature or slightly larger radius of curvature of the corresponding anterior condyles of the femoral component, thereby providing a total knee replacement prosthesis. The prosthesis, if desired, can provide a substitute for the function of the cruciate ligaments, including the function of an anterior and/or a posterior cruciate ligament.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a diagrammatic illustration of a tibial prosthetic knee implant containing two condylar surfaces ((2) and (4)) and an intercondylar projection (6). Anterior condylar surfaces ((8) and (10)) are curved and elevated anteriorly to conform to the anterior femoral component.

FIG. 2 depicts separated femoral and tibial components to illustrate the engaging surfaces on the tibial condyles ((8) and (10)) and on the tibial component projection (12). The femoral component shows the anterior femoral condylar ((16) and (18)) and the intercondylar portion (14).

FIG. 3 illustrates a cross sectional view of the femoral and tibial components articulating with each other in full extension in mid flexion. The intercondylar portion of the femoral component (14) is engaged with the anterior surface of the tibial projection (12), the anterior femoral condylar ((16) and (18)) is slided over the anterior tibial condyles ((8) and (10)).

FIG. 4 depicts an exploded view of the femoral and tibial components, showing a tibial component with a central projection (20) with anterior (22) and posterior (24) surfaces, which articulate with distal intercondylar surface of the femoral component (26) and an intercondylar cam (28).

FIG. 5 illustrates a cross sectional view of the femoral and tibial components, depicting a tibial component with a central projection (20) with anterior (22) and posterior (24) surfaces, which is articulated with distal intercondylar surface of the femoral component (26) and an intercondylar cam (28) during a late flexion.

FIG. 6 shows an exploded view of the secondary articulating surfaces, the femoral and tibial components.

FIG. 7 depicts a cross sectional view of the tibial post and the femoral stop. The stop prevents the femur from displacing posteriorly in full extension, and the anterior intercondylar region of the tibial liner prevents the femur from displacing anteriorly as the femur is flexed.

FIG. 8 shows a cross sectional view of the articulating and the secondary stop surfaces, conforming middle surfaces of tibial lines and the intercondylar groove on the femur.

FIG. 9 shows a superior view of the post, the curvature in the transverse or frontal plane, which allow rotation of the tibia on the femur.

FIG. 10 depicts a cross sectional view of the post.

FIGS. 11-A, 11-B, and 11-C show contact at the tibial-femoral articulation of the anterior cruciate substituting knee at 0, 60, and 90 degrees of flexion, respectively.

FIGS. 12-A, 12-B, and 12-C show contact at the tibial-femoral articulation of the conventional posterior cruciate substituting knee at 0, 60, and 90 degrees of flexion, respectively.

FIG. 13 depicts tibial post forces (shown by arrows) in the anterior cruciate substituting knee in full extension.

FIG. 14 depicts tibial post forces (shown by arrows) in the conventional posterior cruciate substituting knee in full extension.

FIG. 15. Contact stresses on the tibial surfaces show midline contact and anterior post contact in full extension.

FIG. 16. Vector plots of the contact stresses in full extension show post contact stresses below 3 MPa.

FIG. 17 shows bicruciate substituting tibial liner with a post to articulate with the intercondylar portion of the femur.

FIG. 18 depicts different close up views of a bicruciate substituting post.

FIG. 19 shows different views of asymmetric post (medial side is smaller in front-back dimensions than the lateral side to allow femoral component external rotation in flexion).

FIG. 20 depicts anterior cruciate ligament substituting knee with intact posterior cruciate ligament of a low conforming design (shallow dish), with central post that is substantially curved in the sagittal and coronal planes. Arrow indicates the intercondylar region of the femur where the post articulates.

FIG. 21 shows a sketch of a deep dish anterior cruciate substituting knee with frontal femoral cam.

FIG. 22 depicts different views of femoral component that articulates with the bicruciate substituting tibial liner with a posterior cam only.

### DETAILED DESCRIPTION OF THE INVENTION

The invention provides knee replacement prostheses, which can provide a substitute function for the function of the deficient anterior and/or a posterior cruciate ligament. The prosthesis comprises a femoral component, having a pair of condylar surfaces and an intercondylar region, a tibial component having a tibial platform and a bearing component, which articulates with the femoral component, and a protrusion from the bearing component, which articulates with the intercondylar portion of the femoral component so as to displace the tibial component anteriorly in extension, and to substitute the function of the deficient anterior and/or a posterior cruciate ligament while allowing posterior movement of the tibial component in flexion and axial rotational movement between the femur and the tibia. The bearing component of the invention is preferably a non-mobile component by being fixed to the tibial component.

The femoral and the tibial components of a total knee replacement in which the function of the anterior cruciate ligament is impaired or absent has necessitated the provision of the herein described prosthetic knee implant which can provide a replacement for the anterior cruciate ligament. The invention provides a prosthetic knee implant, which, if desired, can provide a substitute function for the anterior cruciate ligament of a prosthetic knee in which the function of the anterior cruciate ligament is impaired or absent. The prosthetic knee implant of the instant invention also prevents the particular relative motion, for example, movement in a paradoxical fashion, experienced between the femoral and the tibial components in an anterior cruciate ligament deficient knee joint.

According to the invention, the function of anterior cruciate ligament in a prosthetic knee implant is provided by a central projection from the intercondylar region of the tibial component, which articulates with the intercondylar surface of the femoral condyle. The two components are shaped in such a way that the femoral intercondylar surface has a radius of curvature at its distal most aspect which is slightly smaller than the radius of curvature of the anterior surface of the tibial projection so as to provide a camming action and displace the femoral condyle anteriorly in full extension. The anterior articular surface of the tibial component is also curved with a radius of curvature of the condylar surfaces, which are about the same radius of curvature or slightly larger radius of curvature as the corresponding anterior condyles of the femoral component so as to displace the femoral component posteriorly as the knee is flexed. During the late stages of flexion the femoral component is further displaced posteriorly by the posterior cruciate ligament. Another aspect of this invention involves substitution of the anterior as well as the posterior cruciate ligaments by providing curvatures to the anterior and posterior surfaces of the post in to which the anterior surface of the distal intercondylar surface and a cam engage respectively. Thus, in late flexion the femoral component is further translated posteriorly engaging the cam with the posterior surface of the tibial projection, while in mid-flexion the femora component is translated posteriorly by the engagement of the anterior condyles of the femoral component with the anterior condyles of the tibial component and in early flexion the femoral component is translated anterior by the engagement of the anterior intercondylar surface of the femoral component with the anterior surface of the tibial projection.

According to the invention, the anterior surface of the femoral component contacts the anterior surface of the post in extension between −30 degrees of extension (i.e. 30 degrees of flexion) to 15 degrees of hyperextension.

The articulation between the post and the femoral component (anteriorly in extension and posteriorly in flexion) controls the anterior-posterior locations of the contact between the weight bearing articulating surfaces of the femoral and the tibial components.

Aspects of the present invention provide variable stops only in late extension and late flexion by the interaction of the tibial post and the intercondylar surfaces of the femur. Thus in most of the mid range of flexion the implant is free to move guided by the ligaments and the articulating surfaces and not by the guide surfaces on the tibia. Besides, the presence of the tibia guide surface, which extends from the front to the back of tibial component, does not allow preservation of the posterior cruciate ligament. The single post of the instant knee prosthesis only occupies the mid-portion of the tibia and does not extend all the way back to the posterior aspect of the tibia; therefore, allows a cut out in the tibial component for preserving the posterior cruciate ligament.

In one aspect, the anterior cruciate substituting total knee of the instant invention has a single projection in the middle and with articulating surfaces on the anterior and posterior aspects (rather than posterior and anterior aspects). These articulating surfaces are much closer to the midline of the tibial liner and do not lead to much tilting of the tibial component which could cause loosening or other problems.

According to the instant invention, the intercondylar region which engages with the tibial intercondylar region of the anterior cruciate substituting knee only extends to mid position of the tibia and does not require excessive bone resection from the femur.

The knee prosthesis of the invention has a variable stop surface on the posterior and aspects of the tibial post which are substantially curved in the sagittal plane to allow anterior-posterior translation of the femoral component during extension and early flexion (that is the post can be anywhere from front to back of the tibial component), but the radius of curvature is determined by the amount of desired anterior-posterior translation. The post has a variable radius of curvature, preferably less than about 10 mm.

The knee prosthesis of the invention, wherein the anterior surface of the post can be offset from the main coronal plane of the post by about 0 to about 20 degrees to control femoral component rotation in extension (i.e., larger front to back dimensions on the lateral aspect for the post than the medial aspect). The anterior surface of the post can be substantially curved in the sagittaly plane to allow anterior-posterior translation of the femoral component during extension and early flexion.

The knee prosthesis according to the invention, wherein the posterior surface of the post can be substantially curved in the sagittaly plane to allow anterior-posterior translation of the femoral component during late flexion. The posterior surface of the femoral component contacts the posterior surface of the post in late flexion, wherein the flexion is between about 80 to about 150 degrees. The posterior surface of the post also can be substantially curved in the coronal plane to allow femoral component internal and external rotation. The posterior surface of the post can be offset from the main coronal plane of the post by about 0 to about 20 degrees to control femoral component rotation in flexion (i.e., larger front to back dimensions on the lateral aspect for the post than the medial aspect).

The knee prosthesis of the invention has a tibial post which preferably has a downward sweep on the anterior posterior aspects and preferably does not have an upward sweep.

Unlike conventional knee prosthesis, which controls the anterior/posterior position of the femoral component relative to the tibial platform at any angle of flexion, the knee prosthesis of the invention controls the early and late flexions only and not in the middle flexion. Laxity of the knee is not required to be less than 3 mm in the case of a flexion of greater than about 60 degrees. Thus, the knee prosthesis of the invention can provide a substitute for the function of the cruciate ligaments, including the function of an anterior and/or a posterior cruciate ligament.

The invention provides methods of repairing a damaged knee of a patient in need by implanting a total knee replacement prosthesis comprising the steps of: (a) providing a femoral component having a pair of condylar surfaces and an intercondylar region; and (b) providing a tibial component having a tibial platform and a bearing component which articulate with the femoral component, wherein a protrusion or a tibial post from the bearing component articulates with the intercondylar portion of the femoral component, wherein the tibial post is substantially curved in the sagittal plane to allow anterior translation of the femoral component during extension and early flexion, wherein anterior surface of the post is curved medial laterally to allow femoral-tibial axial rotation, wherein the femoral and tibial components are shaped in such a way that the femoral intercondylar surface has a radius of curvature at its distal most aspect which is slightly smaller than the radius of curvature of the anterior surface of the tibial projection, thereby providing a camming action, wherein the anterior articular surface of the tibial component is curved with a radius of curvature of the condylar surfaces which are about the same radius of curvature or slightly larger radius of curvature of the corresponding anterior condyles of the femoral component, thereby providing a total knee replacement prosthesis.

In one aspect, the invention provides methods of making a total knee replacement prosthesis comprising: (a) obtaining a femoral component having a pair of condylar surfaces and an intercondylar region; (b) obtaining a tibial component having a tibial platform and a bearing component; (c) articulating the tibial platform and the bearing component with the femoral component; (d) articulating a protrusion or a tibial post from the bearing component with the intercondylar portion of the femoral component; (e) shaping the femoral and tibial components in such a way that the femoral intercondylar surface has a radius of curvature at its distal most aspect which is slightly smaller than the radius of curvature of the anterior surface of the tibial projection, thereby providing a camming action; and (f) curving the anterior articular surface of the tibial component with a radius of curvature of the condylar surfaces which are about the same radius of curvature or slightly larger radius of curvature of the corresponding anterior condyles of the femoral component, thereby providing a total knee replacement prosthesis.

The methods also provide that the anterior surface of the post is offset from the main coronal plane of the post by 0 to 20 degrees to control femoral component rotation in extension, wherein the post can be anywhere from front to back of the tibial component.

According to the methods disclosed herein, wherein the anterior surface of the post is substantially curved in the sagittaly plane to allow anterior translation of the femoral component during extension and early flexion. The posterior surface of the post also is substantially curved in the sagittaly plane to allow posterior translation of the femoral component during late flexion. The posterior surface of the femoral component contacts the posterior surface of the post in late flexion and the flexion is between about 80 to about 150 degrees. The posterior surface of the post can be substantially curved in the coronal plane to allow femoral component internal and external rotations. The posterior surface of the post can also be offset from the main coronal plane of the post by about 0 to about 20 degrees to control femoral component rotation in flexion.

The invention will be understood more fully, while the objects and advantages will become apparent, in the following detailed description of preferred embodiments of the invention illustrated in the accompanying drawing:

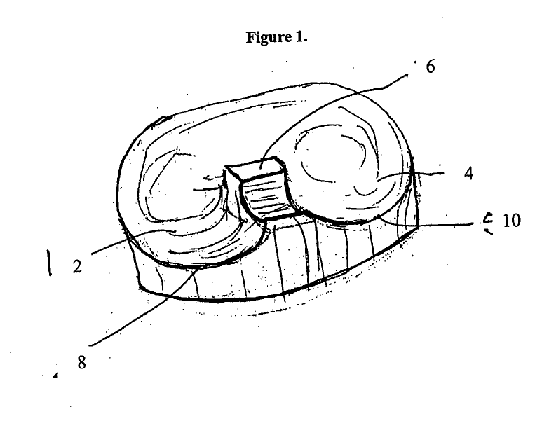

Referring to FIG. 1, a diagrammatic illustration of a tibial prosthetic knee implant constructed in accordance with the present invention. The tibial prosthetic knee implant, according to the invention, has a pair of condylar surfaces (2,4) and an intercondylar projection (6). The anterior surface of the intercondylar surface contains a curvature with either a fixed radius of curvature or a varying radius of curvature and accepts the intercondylar region of the femoral component in full extension and early flexion (flexion angle is approximately 0 to 20 degrees). The anterior condylar surfaces (8,10) also are curved and elevated anteriorly to conform to the anterior femoral component and displace the femur anteriorly in full flexion (flexion angle is approximately 20 to 90 degrees).

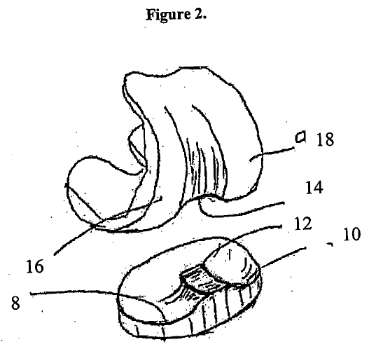

In FIG. 2, the femoral and tibial components of a total knee replacement prosthesis are separated to illustrate the engaging surfaces on the tibial condyles (8,10) and on the tibial component projection (12). Diagrammatically illustrated femoral component shows the anterior femoral condylar (16,18) and the intercondylar portion (14). The intercondylar portion (14) engages on the anterior surface of the tibial projection in full extension and early flexion.

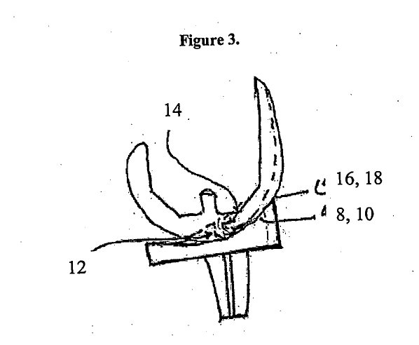

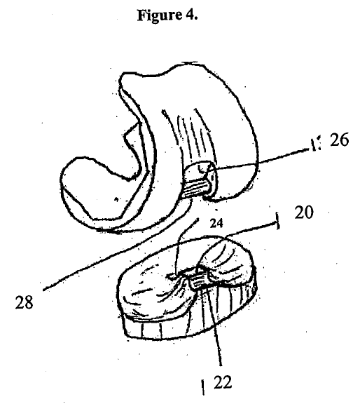

As best seen in FIG. 3, a cross sectional view of the femoral and tibial components articulating with each other in full extension, according to an aspect of the invention. The intercondylar portion of the femoral component (14) engages with the anterior surface of the tibial projection (12) in full extension and displaces the femoral component anteriorly. In mid flexion, the anterior femoral condylar (16,18) slides over the anterior tibial condyles (8,10) and displaces the femoral component posteriorly. The intact posterior cruciate ligament further displaces the femur posteriorly in late flexion (flexion angle is about 80 to about 150 degrees).

Referring now to the drawing, and especially to FIG. 4 thereof, is an exploded view of the femoral and tibial components of an anterior and posterior cruciate substituting total knee prosthesis, showing a tibial component with a central projection (tibial post) (20) with anterior and posterior surfaces (22 and 24, respectively), which articulate with distal intercondylar surface of the femoral component (26) and an intercondylar cam (28). During late flexion the interaction and the posterior surface of the femoral projection and the cam displaces the femoral component further posteriorly in a posterior cruciate deficient or substituting total knee. The anterior surface (22) of the post (20) is curved in the transverse plane, which allows femoral rotation on the tibia.

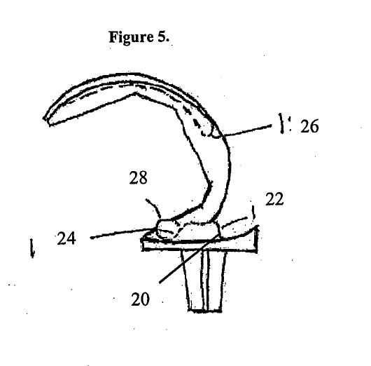

As illustrated somewhat diagrammatically in FIG. 5, is a cross sectional view of the femoral and tibial components of an anterior and posterior cruciate substituting total knee prosthesis, a tibial component with a central projection (20) with anterior and posterior surfaces (22,24), which articulate with distal intercondylar surface of the femoral component (26) and an intercondylar cam (28), respectively. During late flexion the interaction and the posterior surface of the femoral projection and the cam displaces the femoral component further posteriorly in a posterior cruciate deficient or substituting a total knee.

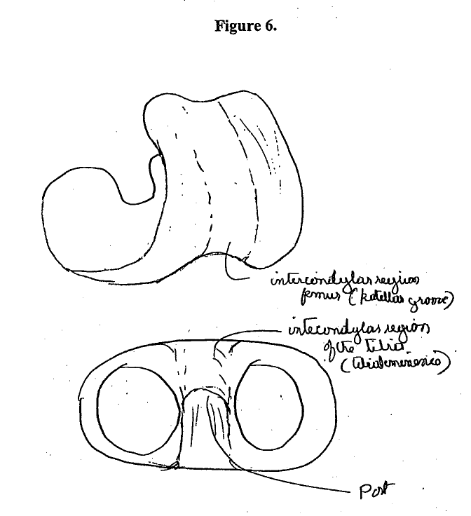

The invention is further depicted in sketches as best seen in FIGS. 6 through 10, which do not limit the invention in any manner.

Secondary articulating surfaces, that is, not the weight bearing surfaces but the variable stop surfaces is depicted in FIG. 6. FIG. 6 also showing the intercondylar femur and tibial regions and relative position of the post.

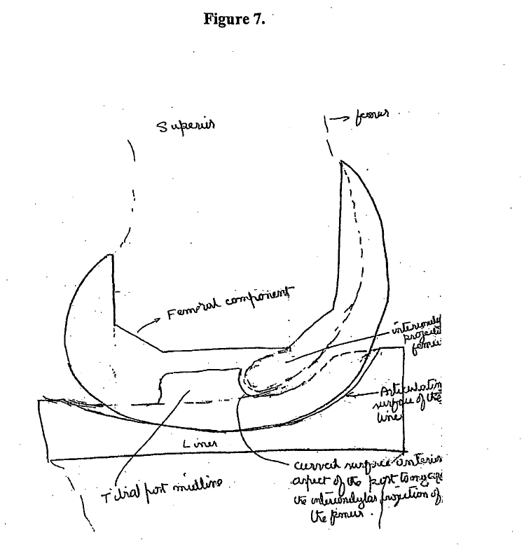

Referring to FIG. 7, depicting a cross sectional view of the tibial post and the femoral stop. It is notable that the stop prevents the femur from displacing posteriorly in full extension, and the anterior intercondylar region of the tibial liner prevents the femur from displacing anteriorly as the femur is flexed. After flexion to about 30 to 60 degrees the femur displaces posteriorly by the action of the posterior cruciate ligament or by a cam posteriorly which articulates with the back surface of the tibial post.

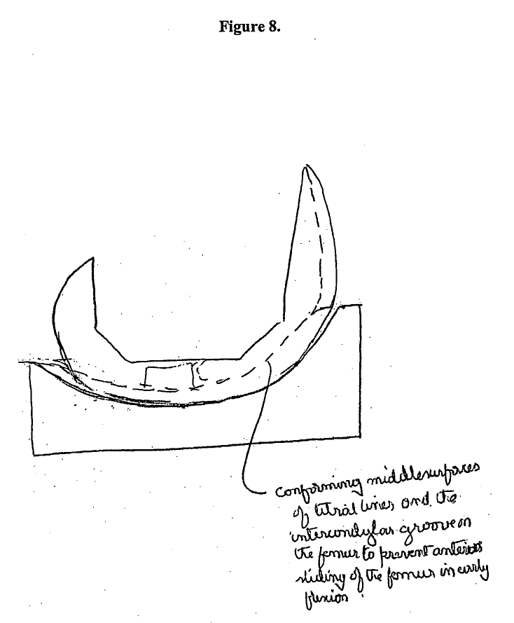

Another cross sectional view of the articulating and the secondary stop surfaces is shown in FIG. 8, which also depicts conforming middle surfaces of tibial lines and the intercondylar groove on the femur to prevent anterior sliding of the femur in early flexion.

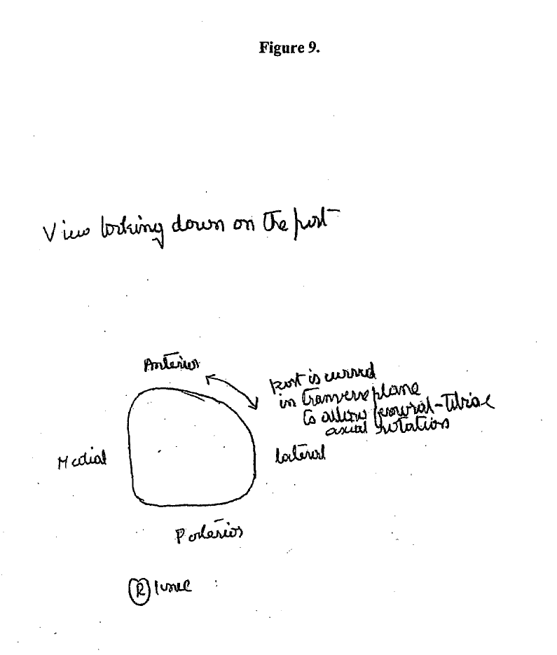

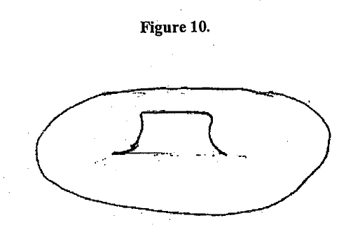

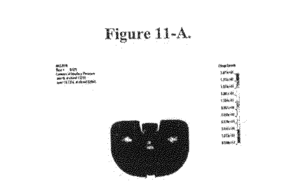

A superior view of the post, as depicted in FIG. 9, is curved in the transverse plane to allow femoral-tibial axial rotation. A cross sectional view of the surface of the post is drawn in FIG. 10.

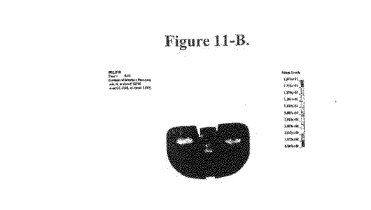

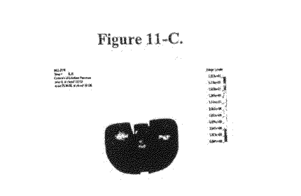

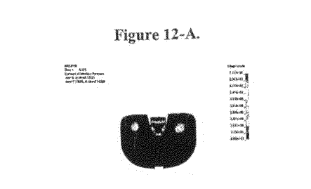

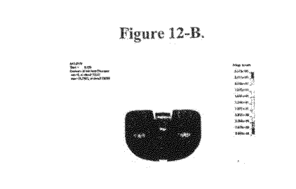

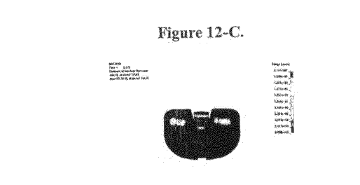

Referring to FIGS. 11 and 12: FIGS. 11-A, 11-B, and 11-C depict contact at the tibial-femoral articulation of the anterior cruciate substituting knee at 0, 60, and 90 degrees of flexion, respectively. The tibial femoral contact areas remain in the middle until about 60 degrees of flexion and then they move posteriorly with further flexion. FIGS. 12-A, 12-B, and 12-C depict contact at the tibial-femoral articulation of the conventional posterior cruciate substituting knee at 0, 60, and 90 degrees of flexion, respectively. The contact areas are located posteriorly at 0 degrees of flexion, move anteriorly at 60 degrees of flexion and then move posteriorly at about 90 degrees of flexion.

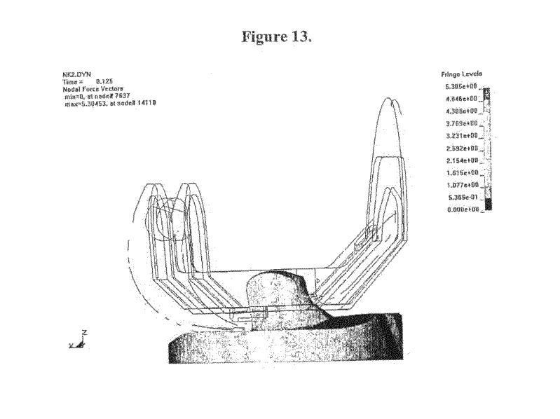

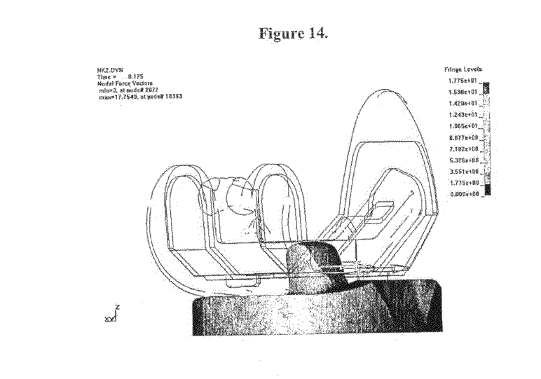

As best seen in FIGS. 13 and 14: the tibial post forces (shown by arrows), in the anterior cruciate substituting knee in full extension are small and distributed evenly on the post (FIG. 13). Whereas, in the conventional posterior cruciate substituting knee in full extension, they are large and localized on the edges of the post (FIG. 14).

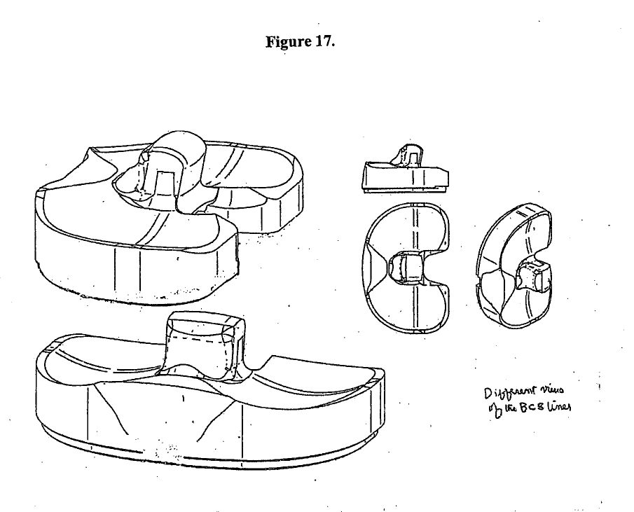

The invention is further depicted in sketches as best seen in FIGS. 17 through 22, which do not limit the invention in any manner.

FIG. 17 depicts bicruciate substituting tibial liner with a post to articulate with the intercondylar portion of the femur, wherein the post is substantially curved in the sagittal and coronal plane to control the antero-posterior. FIG. 17 also depicts displacement and rotation of the femur during flexion-extension.

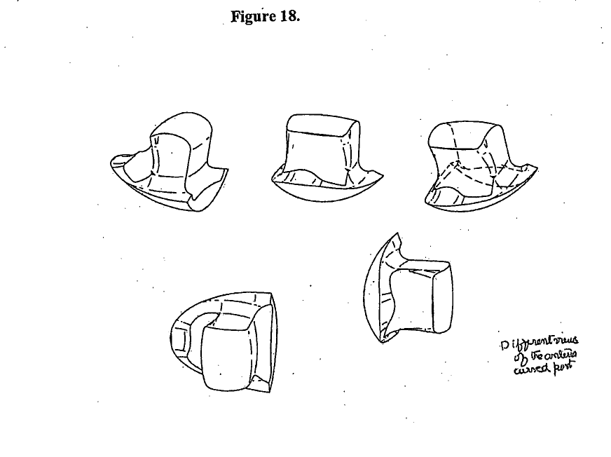

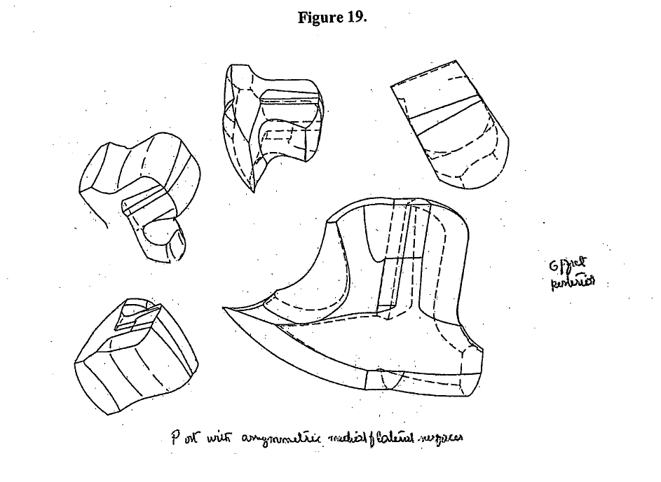

FIG. 18 shows different close up views of the bicruciate substituting post. FIG. 19 shows sketches of asymmetric posts, wherein medial side is smaller in front-back dimensions than the lateral side to allow femoral component external rotation in flexion.

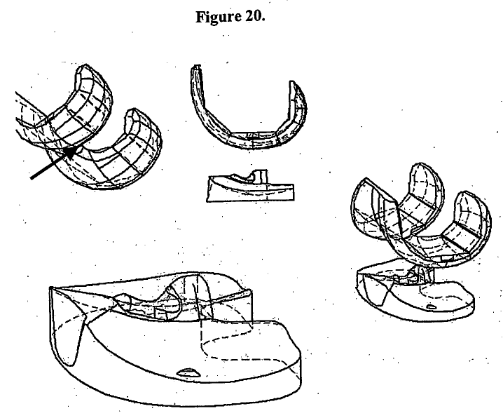

As shown in FIG. 20 are sketches of shallow dish anterior cruciate ligament substituting knee with intact posterior cruciate ligament of a low conforming design, with central post that is substantially curved in the sagittal and coronal planes. The intercondylar region of the femur where the post articulates is indicated by a arrow.

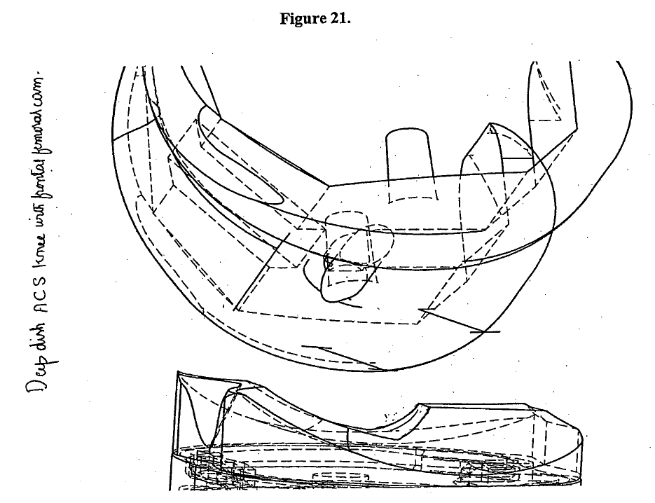

As illustrated in FIG. 21 is sketch of a deep dish anterior cruciate substituting knee with frontal femoral cam. The sketch depicts an anterior cruciate substituting design with intact posterior cruciate ligament employing a cam on the femoral component in the anterior intercondylar area and a post on the liner that is substantially curved in the sagittal plane.

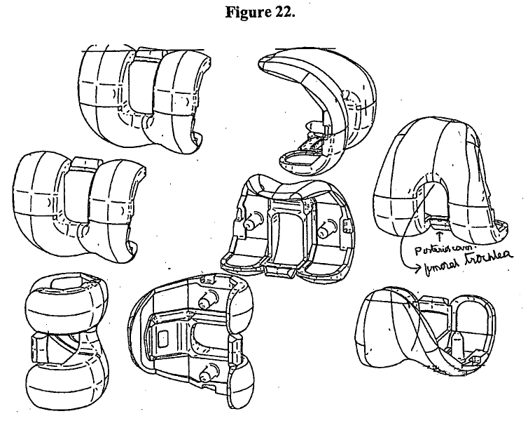

As depicted in FIG. 22, are different views of the femoral component to articulate with bicruciate substituting tibial liner with a posterior cam only.

The products and processes of this invention involve various types of polymeric materials, for example, any polyolefin, including high-density-polyethylene, low-density-polyethylene, linear-low-density-polyethylene, ultra-high molecular weight polyethylene (UHMWPE), or mixtures thereof. Polymeric materials, as used herein, also include polyethylene of various forms, for example, resin powder, flakes, particles, powder, or a mixture thereof, or a consolidated form derived from any of the above.

Ultra-high molecular weight polyethylene (UHMWPE) refers to linear non-branched chains of ethylene having molecular weights in excess of about 500,000, preferably above about 1,000,000, and more preferably above about 2,000,000. Often the molecular weights can reach about 8,000,000 or more. By initial average molecular weight is meant the average molecular weight of the UHMWPE starting material, prior to any irradiation. See U.S. Pat. No. 5,879,400; PCT/US99/16070, filed on Jul. 16, 1999; PCT/US97/02220, filed Feb. 11, 1997; and US Patent publication 20030149125 (U.S. application Ser. No. 10/252,582), filed Sep. 24, 2002.

Crosslinked polymeric material, as used herein, include UHMWPE cross-linked by a variety of approaches, including those employing cross-linking chemicals (such as peroxides and/or silane) and/or irradiation. Preferred approaches for cross-linking employ irradiation. Crosslinked UHMWPE can be obtained according to the teachings of U.S. Pat. No. 5,879,400; U.S. Pat. No. 6,641,617; PCT/US97/02220; and US Patent publication 20030149125 (U.S. application Ser. No. 10/252,582), filed Sep. 24, 2002, the entirety of which are hereby incorporated by reference.

The products and processes of this invention involve various types of metals. The metal can be a cobalt chrome alloy, stainless steel, titanium, titanium alloy or nickel cobalt alloy, for example. Various metal types can also be found in U.S. Ser. No. 60/424,709, filed Nov. 8, 2002 (PCT/US03/18053, filed Jun. 10, 2003, WO 2004000159).

The products of this invention can include an “interface”, which refer as the niche in medical devices formed when an implant is in a configuration where a component is in contact with another piece (such as a metallic or a non-metallic component), which forms an interface between the polymer and the metal or another polymeric material. For example, interfaces of polymer-polymer or polymer-metal are in medical prosthesis, such as knee replacement prostheses. Various metal/non-metal types and interfaces also can be found in U.S. Ser. No. 60/424,709, filed Nov. 8, 2002 (PCT/US03/18053, filed Jun. 10, 2003, WO 2004000159), the entirety of which is hereby incorporated by reference.

In accordance with the invention, the piece forming an interface with polymeric material is, for example, a metal. The metal piece in functional relation with polyethylene, according to the present invention, can be made of a cobalt chrome alloy, stainless steel, titanium, titanium alloy or nickel cobalt alloy, for example.

In accordance with the invention, the piece forming an interface with polymeric material is, for example, a non-metal. The non-metal piece in functional relation with polyethylene, according to the present invention, can be made of ceramic material, for example.

The invention is further described by the following examples, which do not limit the invention in any manner.

### Example 1

### Bicruciate Substituting (BCS) Total Knee

An anterior cruciate ligament substituting knee replacement (bicruciate substituting knee, BCS) was designed by a computer assisted design process using solid modeling software. A tibial liner was designed to have a central post with an anterior surface, which was substantially curved in the sagittal and coronal planes. The curvatures of the anterior surface were designed by subtracting the femoral geometry from the tibial post geometry, with the femoral component in various degrees of flexion, desired anterior-posterior translations, and rotations at different degrees of flexion. The desired anterior-posterior translations and rotations were based on in vivo kinematic data determined using a normal knee. A midline location of femoral tibial articular contact in full extension was desirable, while the contact points were posteriorly with increasing knee flexion. This generated a complex surface geometry with varying degrees of radii in both sagittal and coronal planes for the anterior surface of the post. The simulated kinematics and mechanics were compared to a conventional posterior cruciate (only posterior cruciate substituting knee design (PS design), currently used in patients), which is not designed to substitute the anterior cruciate ligament.

Geometric solid models were created for both types of prosthesis. A bicruciate substituting (BCS) knee replacement prosthesis with a tibial insert containing a central post, which was substantially curved in the sagittal and coronal planes on the anterior surface to articulate with the intercondylar portion of the femoral component in full extension and early flexion (prosthesis of the current design), and a traditional posterior cruciate substituting design (only posterior cruciate substituting design, the PS design) were used to create solid models. The femoral component had a posterior cam in both designs. The tibial liner had the anterior surface, which contacted and articulated with the distal most intercondylar region of the femoral component (trochlea) in full extension, when no external body load was applied, whereas in the PS design no such contact had occurred. A dynamic explicit finite element analysis was carried out for the various activities of daily living such as walking, stair-climbing, and squatting (See, Taylor and Walker, J Biomech. 2001 34(7):839-848; Dennis et al., Clin Orthop. 2003 (416):37-57; and Li et al., 48th Annual Meeting of the OR Society, Poster No: 0967).

A time varying vertical load (acting through the center of the epicondylar axis with a peak load of 1700 Newtons) was used to simulate weight bearing forces. The femoral component was flexed through the center of the epicondylar axis and the load and flexion angle was synchronized with the in vivo data. The femur was allowed to slide in anterior-posterior, medial-lateral and varus-valgus directions without displacement constraints, limited only by the frictional and geometrical forces generated at the contact interfaces. Tibial rotation (internal with increasing flexion) with a maximum of 12 degrees was imposed on the tibial component acting along a vertical axis from the geometrical center of the liner. Contact surfaces were defined at the liner-femoral component and the liner-tibial tray interfaces with static and dynamic frictional coefficients of 0.01. The femoral and tibial components were treated as rigid bodies. UHMWPE liner was modeled as isotropic plastic with material properties measured from earlier experiments (for example, Elastic modulus=820 MPa in the linear region, Poisson ratio=0.439, yield stress=21.78 MPa, hardening modulus=195 MPa). The model contained approximately 180,000 elements but no ligaments or other soft tissue restraints.

The computer simulation study showed that as soon as the loading began in the BCS as designed, there was contact between the anterior surface of the post and the most distal part of the intercondylar region of the femoral component (trochlea). With further load this forced the femoral condyles to articulate with tibial condyles at the center of the tibial condyles medially and laterally. (see FIGS. 11A, 11-D and 11-C). The resulting stresses on the anterior surface of the post were small, <10 MPa, and distributed over a large area of the surface (see FIG. 13). With further flexion of the femoral component, the contact between the femoral condyles and the tibial condyles remained in the center both medially and laterally to about 80 degrees of flexion. Thereafter, contact between the post and the femoral cam occurred on the posterior surface and forced the femur to translate posteriorly resulting in progressive posterior displacement of the femoral tibial condyle contact points medially and laterally till maximum knee flexion.

In contrast, in the traditional PS knee design, no contact occurs between the femoral trochlea and the anterior flat surface of the post at full extension and at the beginning of loading. As the vertical load increased prior to the initiation of flexion, the femoral component slid posteriorly on the tibial liner to the lowest point on the articulation, which was 4 millimeters posterior to the midline. Anterior contact occurred at this point between the post and the femoral component trochlea. However, by then the contact area between the femoral and tibial condyles was several millimeters posterior to the midline (see FIGS. 12-A, 12-B, and 12-C). The contact between the anterior surface of the post and the femoral trochlea also was highly localized to medial and lateral corners of the post. The high localized contact stresses occurred at these regions were about >15 Mpa (see FIG. 14). The contact occurred from the posterior sliding of the femoral component on the tibia prior to any knee flexion, even though the femoral component was oriented in line with the femoral axis and the tibial component did not have any posterior slope. As the femur is flexed under load, the femoral component is translated anteriorly with respect to the tibia. The tibial femoral condylar contact stresses was moved anteriorly reaching 7 millimeters anterior to the midline at 60 degrees of flexion. At about 70 degrees of flexion, contact occurred between the posterior surface of the post and the femoral cam. This led to a rapid posterior translation of the femur with further flexion. The tibial femoral condylar contact surfaces are translated a total of 22 millimeters posteriorly with further flexion to 150 degrees (from about 7 millimeters anterior to the midline to about 15 millimeters posterior to the midline) (see FIGS. 12-A, 12-B, and 12-C).

Therefore, according to the above Anterior Cruciate ligament substituting knee replacement design, the femoral tibial condylar contact points moved in a desirable manner similar to the normal knee. In contract, in the traditional posterior cruciate ligament substituting only total knee replacement design, much larger and paradoxical translations of the contact points occurred. The large translations of the contact points is detrimental to the wear of the plastic, increases the demands on the muscles around the knee, and produces abnormal movements of the total knee replacements. The smaller and more evenly distributed contact stresses between the femoral trochlea and the anterior post surface with the BCS design also are beneficial in decreasing the chances for tibial component breakage. Minimizing the large translations of the contact areas also minimizes the tilting the liner within the metal tray and reduces the shear and tensile forces at the prosthesis-bone interface, thereby improving the longevity of total knee replacements.

### Example 2

### Anterior Cruciate Substituting Knee with an Intact Posterior Cruciate

An anterior cruciate ligament substituting knee replacement (anterior cruciate substituting knee with an intact posterior cruciate) was designed. A tibial liner was designed with medial and lateral condyles with radii of curvatures slightly larger than the radii of curvatures of the femoral component in the coronal and sagittal planes. A central post was added to the tibial liner with an anterior surface, which was substantially curved in the sagittal and coronal planes. In order to do this, a large box shaped post was added to the tibial liner in the intercondylar region. The femoral component was then placed on the tibial liner with varying degrees of flexion and desired anterior-posterior translations and rotations to simulate the kinematics of the normal knee in flexion. The femoral geometry was then subtracted from the tibial liner post at the different degrees of femoral component position. The curvatures of the anterior surface of the post were designed by subtracting the femoral trochlear geometry from the tibial post geometry, with the femoral component in various degrees of flexion (from full extension to 30 degrees of flexion) and desired anterior-posterior translations and rotations at different degrees of flexion. The desired anterior-posterior translations and rotations were based on in vivo kinematic-data determined using a normal knee. A midline location of femoral tibial articular contact in full extension was desirable, while the contact points were posteriorly with increasing knee flexion. This generated a complex surface geometry with varying degrees of radii in both sagittal and coronal planes for the anterior surface of the post. The posterior surface of post did not make contact with the femoral component. The posterior cruciate ligament was intact and provided the posterior translation and rotation of the femoral component greater than 30 degrees of flexion.

A dynamic explicit finite element analysis also was carried out for this design for the various activities of daily living such as walking, stair-climbing, and squatting (See, Taylor and Walker, J Biomech. 2001 34(7):839-848; Dennis et al., Clin Orthop. 2003 (416):37-57; and Li et al., 48th Annual Meeting of the OR Society, Poster No: 0967).

The femoral component flexion and rotation as well as the external loads were input in the model, but the anterior-posterior displacement of the femoral component were not constrained. The results of the analysis showed that during full extension, the contact points between the femur and the tibial liner were maintained near the middle of the tibial condyles during the first thirty degrees of flexion. During full extension the femoral trochlea contacted the anterior surface of the post but contact stresses on the post were modest and remained below 3 MPa, which was desirable for the post. Initial posterior contact of the femoral component on the tibial condyles and the subsequent paradoxical anterior translation of the femoral component during flexion did not occur, therefore providing a more normal kinematics to the knee.

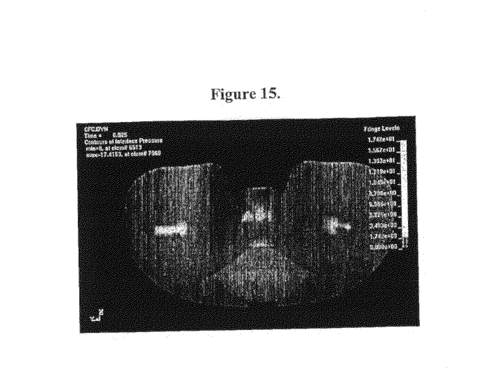

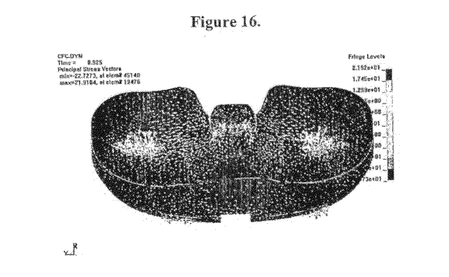

Contact stresses on the tibial surfaces, according to the above study, show midline contact and anterior post contact in full extension (see FIG. 15). Vector plots of the contact stresses in full extension indicate that post contact stresses remained below 3 MPa (see FIG. 16).

### Example 3

### Anterior Cruciate Substituting Knee with an Anterior Femoral Cam and Intact or Absent Posterior Cruciate

An anterior cruciate ligament substituting knee replacement (anterior cruciate substituting knee with an intact or absent posterior cruciate ligament and an anterior femoral cam) was designed. The tibial liner was deeply dished medial and lateral condyles with radii of curvatures slightly larger than the radii of curvatures of the femoral component in the coronal and sagittal planes. The anterior articular surface of the tibial liner was further elevated to conform with the anterior surface of the femoral component to prevent additional resistance to the anterior translation of the femoral component in mid and late flexion. An anterior cam was added to the femoral component near the trochlear region attaching the medial and lateral femoral condyles anteriorly. A central post was added to the tibial liner with an anterior surface, which was substantially curved in the sagittal plane. In order to do this, a large box shaped post was added to the tibial liner in the intercondylar region. The femoral component was then placed on the tibial liner with varying degrees of flexion and desired anterior-posterior translations and rotations to simulate the kinematics of the normal knee in flexion. The curvatures of the anterior surface of the post were designed so that the femur extends from 30 degrees of flexion, the cam and post contact displace the femoral component anteriorly to the midline, even if the femoral-tibial condylar contact at 30 degrees occurs posteriorly. The desired anterior-posterior translations and rotations were based on in vivo kinematic data determined using a normal knee. A midline location of femoral tibial articular contact in full extension was desirable, while the contact points were posteriorly with increasing knee flexion. The posterior surface of the post did not make contact with the femoral component. The posterior cruciate ligament was intact and provided the posterior translation and rotation of the femoral component greater than 30 degrees of flexion. No such posterior translation would take place if the posterior cruciate is deficient and the stability in flexion is provided by the conformity between the femoral and tibial condylar surfaces.

A dynamic explicit finite element analysis also was carried out for this design for the various activities of daily living such as walking, stair-climbing, and squatting (See, Taylor and Walker, J Biomech. 2001 34(7):839-848; Dennis et al., Clin Orthop. 2003 (416):37-57; and Li et al., 48th Annual Meeting of the OR Society, Poster No: 0967). The femoral component flexion and rotation as well as the external loads were input in the model, but the anterior-posterior displacement of the femoral component were not constrained. The results of the analysis showed that during full extension, the contact points between the femur and the tibial liner were maintained near the middle of the tibial condyles or slightly posterior during the first thirty degrees of flexion. From thirty degrees of flexion to full extension, the femoral cam contacted the anterior surface of the post gradually displacing the femoral component condylar contact point anteriorly to the midline. The contact stresses on the post were modest and remained below 5 MPa, which was desirable for the post. Initial posterior contact of the femoral component on the tibial condyles and the subsequent paradoxical anterior translation of the femoral component during flexion did not occur, therefore providing a more normal kinematics to the knee.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A knee replacement prosthesis comprises:
(a) a femoral component having a pair of condylar surfaces and an intercondylar region; and
(b) a tibial component having a tibial platform and a bearing component which articulate with the femoral component, wherein a protrusion or a tibial post from the bearing component articulates with the intercondylar portion of the femoral component, wherein the tibial post is substantially curved in the sagittal plane to allow anterior-posterior translation of the femoral component during extension and early flexion, wherein anterior and posterior surfaces of the post is curved to allow and control femoral-tibial axial rotation.

2. The knee replacement prosthesis of claim 1, wherein the post can be anywhere from front to back of the tibial component.

3. The knee replacement prosthesis of claim 1, wherein the anterior surface of the post is substantially curved in the sagittaly plane to allow anterior translation of the femoral component during extension and early flexion.

4. The knee replacement prosthesis of claim 1, wherein the anterior surface of the femoral component contacts the anterior surface of the post in extension and early flexion wherein the flexion is between about 0 to about 20 degrees.

5. The knee replacement prosthesis of claim 1, wherein the posterior surface of the post is substantially curved in the sagittaly plane to allow posterior translation of the femoral component during late flexion.

6. The knee replacement prosthesis of claim 1, wherein the posterior surface of the femoral component contacts the posterior surface of the post in late flexion, wherein the flexion is between about 80 to about 150 degrees.

7. The knee replacement prosthesis of claim 1, wherein the posterior surface of the post is substantially curved in the coronal plane to, allow femoral component internal and external rotation.

8. The knee replacement prosthesis of claim 1, wherein the posterior surface of the post is offset from the main coronal plane of the post by about 0 to about 20 degrees to control femoral component rotation in flexion.

9. The knee replacement prosthesis of claim 1, wherein the replacement prosthesis is a substitute for the function of an anterior and/or a posterior cruciate ligament.

10. The knee replacement prosthesis of claim 1, wherein the bearing component is non-mobile.

11. The knee replacement prosthesis of claim 1, wherein the bearing component is mobile.

12. The knee replacement prosthesis of claim 1, wherein the post has a variable radius of curvature of less than about 10 mm.

13. The knee replacement prosthesis of claim 1, wherein the anterior surface of the post is offset from the main coronal plane of the post by 0 to 20 degrees to control femoral component rotation in extension.

14. The knee replacement prosthesis of claim 1, wherein the tibial post which has a downward sweep on the anterior posterior aspects.

15. The knee replacement prosthesis of claim 1, wherein the knee controls anterior/posterior position of the femoral component relative to the tibial platform at early and late flexions only and not in the middle flexion.

16. The knee replacement prosthesis of claim 1, wherein anterior surface of the intercondylar surface has a fixed or variable-radius of curvature.

17. The knee replacement prosthesis of claim 1, wherein anterior surface of the intercondylar surface accepts the intercondylar region of the femoral component in full extension and early flexion.

18. The knee replacement prosthesis of claim 17, wherein the flexion is about 0 to about 20 degrees.

19. The knee replacement prosthesis of claim 1, wherein anterior condylar surfaces of the tibial component are curved and elevated anteriorly to conform to the anterior femoral component and displace the femur anteriorly in extension and early flexion.

20. The knee replacement prosthesis of claim 19, wherein the flexion is about 0 to about 20 degrees.

21. The knee replacement prosthesis of claim 1, wherein the intercondylar portion of the femoral component engages the protrusion from the bearing component in full extension and early flexion.

22. The knee replacement prosthesis of claim 1, wherein, at a mid flexion, anterior femoral condylar of the femoral component slides over anterior tibial condyles of the tibial component and displaces the femoral component posteriorly.

23. The knee replacement prosthesis of claim 1, wherein a central projection of the tibial component articulate with distal intercondylar surface of the femoral component or an intercondylar cam.

24. The knee replacement prosthesis of claim 1, wherein intact posterior cruciate ligament displaces femur posteriorly in late flexion.

25. The knee replacement prosthesis of claim 24, wherein the flexion is about 80 to about 150 degrees.

26. The knee replacement prosthesis of claim 1, wherein the anterior surface of the post is curved medial laterally to allow femoral-tibial axial rotation, wherein the femoral and tibial components are shaped in such a way that the femoral intercondylar surface has a radius of curvature at its distal most aspect which is slightly smaller than the radius of curvature of the anterior surface of the tibial projection, thereby providing a camming action, wherein the anterior articular surface of the tibial component is curved with a radius of curvature of the condylar surfaces which are about the same radius of curvature or slightly larger radius of curvature of the corresponding anterior condyles of the femoral component.

27-71. (canceled)
